(19) United States
(12) Patent Application Publication
(10) Pub. No.: US 2022/0378681 A1
(43) Pub. Date: Dec. 1, 2022

(54) SKIN CARE FORMULATION WITH LIPOPHILIC PEPTIDES

(71) Applicant: DECIEM BEAUTY GROUP INC., Toronto (CA)

(72) Inventors: Alejandro Saldarriaga LONDONO, Toronto (CA); Zevena Pratiwi TEO, Toronto (CA); Ruth Naomi Limcangco JAYME, Toronto (CA); Prudvi Mohan KAKA, Markham (CA)

(21) Appl. No.: 17/769,681

(22) PCT Filed: Oct. 15, 2020
(86) PCT No.: PCT/CA2020/051380

§ 371 Date: Apr. 15, 2022

Related U.S. Application Data
(60) Provisional application No. 62/916,900, filed on Oct. 18, 2019.

(51) Int. Cl. A61K 8/64; A61Q 19/00; A61K 8/37; A61K 8/31; A61K 8/60; A61K 8/9706; A61K 8/9794; A61K 8/49

(52) U.S. Cl. CPC A61K 8/64; A61Q 19/00; A61K 8/375; A61K 8/31; A61K 8/602; A61K 8/9706; A61K 8/9794; A61K 8/4973; A61K 2800/48; A61K 2800/524; A61K 2800/522

(57) ABSTRACT

A novel cosmetic skin care composition is provided which comprises a solubilized lipophilic peptide, preferably palmitoyl peptide, preferably palmitoyl-GHK-Cu, at a concentration in the range of 0.2-1.1%, the formulation being substantially free of alcohols or preservatives. A method of formulating the composition is also provided to provide a high concentration of the peptide in the final composition.

### FIELD OF THE INVENTION

The present invention generally relates to the field of personal care formulations for the skin, and more particularly to a skin care composition having a particularly high concentration of a lipophilic peptide, as well as a method of formulating said composition.

### BACKGROUND OF THE INVENTION

Peptides are naturally occurring short amino acid chains which have been found to be important in many cellular processes relating to skin regeneration, including in the stimulation of collagen synthesis. It has also been well-established that many peptides can be topically applied and achieve some penetration of the epidermal barrier. For the past several years, there have been many topical cosmeceuticals made available which incorporate peptides, and which have been promoted as providing anti-aging effect for the skin.

Copper Tripeptide-1 (also referred to as “GHK-Cu”) is a small peptide composed of three amino acids, namely, glycine, histidine, and lysine, which form a complex with the physiologically beneficial mineral, copper. GHK-Cu occurs naturally in the body, and has also been synthesized for inclusion in cosmeceuticals. It is a member of the matrikine peptide family, and is well-established as an efficacious ingredient in skin care formulations. This peptide has been observed to improve skin elasticity and firmness, reduce wrinkling, and increase skin clarity. GHK-Cu is hydrophilic but of limited solubility, and has typically been incorporated into cosmetic formulations in solution form at percentages in the range of 0.02-0.1%.

Other categories of peptides have been developed which also incorporate a fatty acid side chain, most commonly a palmitoyl group. Examples are palmitoyl pentapeptide-4, palmitoyl hexapeptide-12, and palmitoyl-lysyl-threonyl-threonyl-lysyl-serine. Such peptides incorporating a palmitoyl chain are lipophilic and hydrophobic, and tend to exhibit superior delivery across the epidermal barrier when compared to the corresponding free peptides. This is because palmitoylation generates sufficient hydrophobicity to confer a strong affinity to cellular membranes, promoting interaction with the lipid bilayer and localization of the peptide to the membrane's surface. The palmitoyl group thereby serves as a lipid anchor but is also believed to play a role in localizing the peptide to functional membrane subdomains. Therefore, palmitoylation increases the penetration and therefore the efficacy of the corresponding peptide.

A preferred peptide for use in the formulation of the invention is palmitoyl-GHK-Cu (“Pal-GHK-Cu”), which combines the benefits of the GHK-Cu peptide with a palmitoyl side-chain, causing the peptide to become hydrophobic. Although Pal-GHK-Cu has been commercially available for several years, the inventors know of no cosmeceutical formulations which have been marketed using this ingredient.

The typical manner in the art to solubilize a hydrophobic peptide would involve the use of alcohols as solvents, such as methanol. However, alcohols or other typical solvents are generally undesirable for inclusion in high amounts in skin care formulations, as they are drying for the skin, which would counteract the beneficial effects of the peptide. The lack of an alcohol-free method of solubilizing lipophilic peptides such as Pal-GHK-Cu may be a reason why it has not yet been commercially used in anti-aging skin formulations.

As well, most lipophilic peptides, including Pal-GHK-Cu, are difficult to get into solution at higher levels than about 0.02-0.1% of the final formulation. At concentrations higher than this range, the formulations tend to become “suspensions” of the peptide rather than “solutions”. As with topical pharmaceuticals generally, the active substance must be in solution in order to be optimally absorbed by the skin. Topical suspensions are generally less efficacious and slower-acting when compared to solutions, as the solid masses of peptide would be required to first disintegrate and disaggregate prior to absorption by the skin.

Peptides are also fairly delicate molecules, being sensitive to degradation when in solution. It has therefore been typically necessary to include significant amounts of preservatives in the formulation. However, high levels of preservatives are known to disrupt the natural microbiota of the skin, and some users have sensitivities and allergies to preservatives. As well, consumers are becoming ever more conscious of extraneous ingredients in their personal care products, so it would be advantageous to formulate a peptide-containing cosmeceutical which maximizes the proportion of active ingredients, with extraneous preservatives being made optional to the formulation, while maintaining acceptable shelf life and stability for the product.

In this respect, it would also be desirable to provide a cosmetic formulation in which lipophilic peptides such as Pal-GHK-Cu are incorporated in solution form at higher concentrations than are typically available in the field, and which is substantially free of harsh solvents or alcohols, and with preservatives being optional to the formulation.

### SUMMARY OF THE INVENTION

A novel cosmetic skin care composition has now been developed which includes a lipophilic peptide at relatively high concentrations in the range of 0.2 to 1.1%, preferably at a level of 1.0%. The formulation does not include the use of an alcohol as a solvent, and preservatives are not necessary to the formulation. The formulation further constitutes a solution of the peptide rather than a suspension.

Preferred types of lipophilic peptides include those containing a palmitoyl group. A particularly preferred peptide for use in this invention is Pal-GHK-Cu. The formulation of the invention may also be prepared using another lipophilic peptide, Pal-GHK.

A method of formulating the composition is also provided which resolves the solubility difficulties associated with lipophilic peptides such as Pal-GHK-Cu, while also largely avoiding the use of harsh solvents and alcohols.

Accordingly, in one aspect of the invention, a formulation is provided comprising a lipophilic peptide which is incorporated in solution at levels of 0.2-1.1%, preferably 1.0%.

In another aspect of the invention, a formulation is provided comprising a lipophilic peptide which is pre-dissolved in a glycolipid mix, said formulation also comprising one or more additional cosmetic ingredients.

In another aspect of the invention, a formulation is provided comprising a lipophilic peptide such as Pal-GHK-Cu combined with glycolipids, and further combined with additional oils and a sucrose base which results in a gel formulation that has low water activity, and which therefore does not require the incorporation of extraneous preservatives.

In a further aspect of the invention, a formulation is provided comprising a lipophilic peptide combined with glycolipids and further combined with additional skin conditioning actives, which forms a transparent or translucent gel formulation that transforms into a substance having a creamy emollient feel upon addition of body temperature-level heat and application to the skin.

In a still further aspect of the invention, a formulation is provided comprising lipophilic peptides such as Pal-GHK-Cu or Pal-GHK combined with glycolipids, and skin conditioning actives, in gel form, which is suitable as a skin care formulation, being both non-sensitizing and non-comedogenic.

In a still further aspect of the invention, a method of formulating a skin care composition having a high concentration of at least one lipophilic peptide, and substantially no alcohols is provided.

In a still further aspect of the invention, a method of formulating a skin care composition having a high concentration of at least one lipophilic peptide, and substantially no preservatives is provided.

There are a number of advantages to the present invention. The present cosmetic composition provides the consumer with a skin care formulation containing very high levels of lipophilic and efficacious peptides that facilitates skin renewal and therefore a more youthful appearance. The peptide is further combined with a number of other skin care actives to increase the benefit to the skin. In the base formulations described, almost all of the ingredients used provide some benefit to the skin.

These and other aspects of the invention will become apparent from the description that follows.

### DETAILED DESCRIPTION OF THE INVENTION

Provided herein is a novel skin-care formulation containing a lipophilic peptide that has been incorporated at approximately a ten-fold higher concentration than has been achieved for a peptide solution when incorporated into a cosmetic formulation, which provides for increased levels of peptide for topical delivery to the user's epidermal cell layers to facilitate skin regeneration.

While there are some cosmetic formulations that may claim higher levels of lipophilic peptides than this range, to the best of the inventors' knowledge, those are peptides provided as suspensions, not in solution. Commercially available formulations incorporating peptides in solution rather than as a suspension have been provided in the range of 0.02-0.1%. It is advantageous to completely solubilize peptides as they are thus poised to immediately provide benefits to the skin versus requiring further processing prior to their action.

The formulation of the invention is substantially free of alcohols and other solvents, such as methanol, pentylene glycol, propylene glycol, 1,3-butanediol, triclosan, toluene, dimethyl isosorbide, and diethylene glycol monoethyl ether. Preservatives are entirely optional to the formulation. If Pal-GHK-Cu is used, the completed product forms a transparent blue gel which, upon addition of body heat and application to the skin, transforms into a creamy textured emulsion that absorbs readily into the skin without any oily or greasy feel. If Pal-GHK is used, the gel formed is translucent and lacks the blue colour, but is otherwise similar in consistency to the transparent gel described above. In either case, the products are further suitable for use as commercial beauty products, having adequate shelf life and being both non-sensitizing and non-comedogenic.

Preferred types of lipophilic peptides for use in the invented formulation and with the described formulation methods are palmitoylated peptides. A particularly preferred palmitoylated peptide is Pal-GHK-Cu, which has heretofore not been successfully incorporated in any skin care formulations known to the inventors. Pal-GHK has been more extensively used in various applications, but to the best of the inventors' knowledge, never to the levels above 0.1% of the final formulation by weight, and in solution.

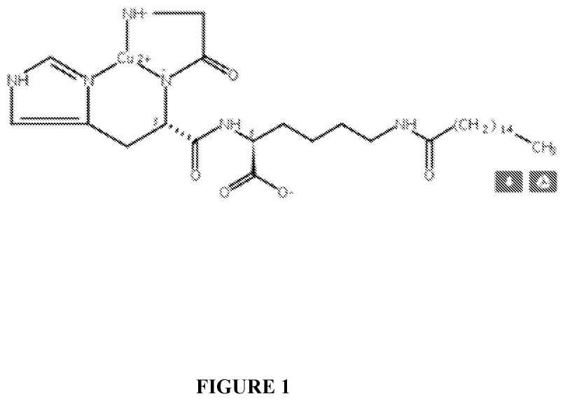

The structure for Pal-GHK-Cu is shown in FIG. 1. GHK corresponds to glycine-histidine-lysine peptide, which is bonded to a copper ion and to a palmitoyl group, C₃₀H₅₁CuN₆O₅, which may also be expressed as Cuprate (1-), [glycyl_{K}N-L-histidinyl-_{K}N, _{K}N—N-(1-oxohexadecyl)-L-lysinato(3-)].

The Pal-GHK-Cu used is provided as a blue powder by NEORE® Pharmaceutical Group Co., Ltd. Although copper ions normally present as a green colour, when complexed with peptides they form a vibrant blue colour, which provides an added visual benefit and appeal of using this particular peptide.

The Pal-GHK used was also provided by NEORE Pharmaceutical Group Co., Ltd. This ingredient presents as a fine white powder.

Embodiments of the present invention are described as follows, which should not to be construed as limiting.

### Formulation Method

For formulating compositions of the present invention, most industrial homogenizing mixers (“kettles”) which are sized and configured to mix personal care formulations will be suitable. The kettle needs to be equipped with standard features such as interior side wall scrapers (also called sweepers) that are separately controllable from the homogenizing paddles, and the kettle also requires a valve at the bottom for taking test samples. The kettle should be jacketed as well so that the interior temperatures of the mixture can be cooled when necessary. While all of the formulation steps may take place in a room temperature environment, mixtures in kettles typically heat up as mixing proceeds over extended periods. It is therefore necessary to cool the mixtures especially at the later stages of mixing in order to maintain room temperature levels inside the kettles. This is particularly important when working with delicate ingredients such as peptides.

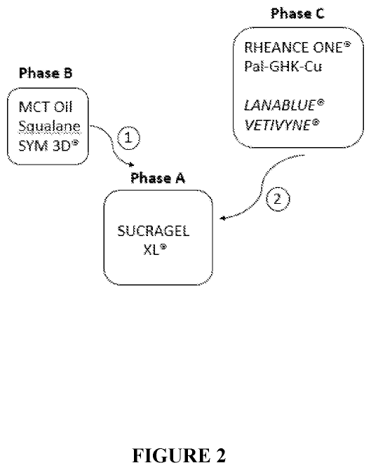

A formulation of the invention can be prepared in three separate core phases (Phases A, B, and C), with optional additional ingredients for some of the phases, as described below. If a preservative is included, it may be added in an additional Phase D. An overview of the core process of combining phases A, B, and C is also shown in FIG. 2.

All amounts for each ingredient are provided as percentage of weight in the final formulation, unless otherwise noted.

In phase C, the lipophilic peptide becomes solubilized. As mentioned above, there are challenges in solubilizing lipophilic peptides without using alcohols, particularly when higher concentrations are sought. Several solubilizers were tried without success, but the inventors found that a glycolipid solution provided good results with high levels of solubility up to a level of 1.1% for lipophilic peptides. This was surprising as glycolipid solutions are generally recommended and used for foaming formulations such as toothpastes, cleansing foams, body washes, and shampoos. Foaming is not a desirable characteristic for a peptide formulation, and it was not expected that the glycolipid solution would successfully solubilize a powdered lipophilic peptide. However, the inventors discovered that glycolipids are indeed effective to solubilize powdered lipophilic peptides at relatively high concentrations.

After trying other solubilizers, the inventors found success in using a proprietary sucrose-derived glycolipid solution supplied by EVONIK® Nutrition & Care GmBH, marketed under the brand RHEANCE ONE®, its INCI identification being “glycolipids”. This ingredient is promoted as a glycolipid solution (50% w/v), is alcohol-free, and presents as a low viscosity, amber-coloured liquid. It is added to the homogenizer first in an amount which will result in levels of between 1-6% by weight in the final formulation. Preferred amounts for the glycolipid solution in the final formulation are in the range of 1.25-5%. The glycolipid solution is agitated for 20 minutes at room temperature at a speed of approximately 3,000 RPM, with additional side sweeps using side wall scrapers at a speed of 10 RPM. Such speeds of mixing have been found to result in suitable agitation without foaming, and also adequately prepares the glycolipid solution to accept dissolution of additional ingredients.

After 20 minutes of agitation as outlined above, a quantity of lipophilic peptide such as Pal-GHK-Cu may then be added to the glycolipids. Pal-GHK-Cu presents as a fine blue powder and was supplied by NEORE® Pharmaceutical Co., Ltd. Amounts of Pal-GHK-Cu may be provided in an amount such that resulting levels of 0.2-1.1% by weight in the final formulation can be achieved, which is a much higher percentage range compared to formulations of the prior art, which incorporate a maximum of 0.1% in solution. When using the specified glycolipids mixture in the above conditions as the base for solubilizing this peptide, a higher level of solubilization can be achieved. A preferred amount of Pal-GHK-Cu is 1% by weight in the final formulation.

When working with powdered peptides, due to the nature of the ingredients as fine powders, it is also important to visually inspect the vessel to ensure that all powder has been incorporated and that excess powder is not adhered to the walls of the vessel. During the course of mixing, periodic samples of the glycolipids mixture with the peptide incorporated should be taken from the bottom of the kettle, where undissolved solids tend to aggregate. This process of sampling is called a valve purge or a bucket flip. The sample should be centrifuged to confirm complete dissolution.

Once it has been confirmed that the lipophilic peptide has been sufficiently incorporated and that the solution is homogenous, which is typically after 3-5 hours of agitation at 3000 RPM with a side sweep speed of 10 RPM, phase C is complete. It can be set aside and maintained at low RPM in the range of 500 RPM until ready for mixing with phases A and B described separately below.

Optional skin care actives such as other botanical ingredients may also be incorporated into phase C. For instance, LUCAS MEYER COSMETICS® offers a suitable algal extract marketed under the trademark LANABLUE PARABEN FREE® (“Lanablue”), which contains Aphanizomenon flos-aquae var. flos aquae extract, combined with water and small amounts of hydrogenated starch hydrolysate (a skin conditioner), phenoxyethanol (a preservative), and potassium sorbate (a preservative). Lanablue has been promoted as reducing the appearance of wrinkles and providing a smoother appearance to the skin, through the algal extracts which are said to provide the benefits of retinoid-like activity but without the side effects associated with retinoid use.

If used, Lanablue has been successfully incorporated at the first step of Phase C, being added to the mixing vessel at the same time as the glycolipids. The Lanablue can then be mixed into solution using the same conditions outlined above. An amount of Lanablue suitable for use in the formulation of the invention may range from 3-5% by weight in the final formulation. In the example formulations, an amount corresponding to 5% was used.

An additional optional skincare active ingredient that may be incorporated into Phase C is an extract of the roots of the vetiver plant (also known as Vetiveria zizanioides, Chrysopogon zizanioides, Andropogon squarrosus, or Andropogon muricatus), a perennial grass native to India. Vetiver has been used as a source for essential oils in the manufacture of fine fragrances. In addition to its pleasant fragrance, extracts of vetiver root have been found to provide additional benefits as a general skin conditioner, as they function to improve skin hydration, reduce wrinkles, and increase skin firmness. A suitable vetiver extract is offered by GIVAUDAN® under the trademark VETIVYNE®. Amounts corresponding to 1-3% VETIVYNE by weight in the final formulation can be successfully used. The amount used in the example formulations corresponded to a final percentage of 3%.

If used, VETIVYNE may be incorporated into Phase C during the 20 minute block of mixing at 3,000 RPM with a side sweep speed of 10 RPM. A convenient time to incorporate VETIVYNE is at approximately 10 minutes into the 20 minute block of mixing time.

Another optional ingredient is a film former, which may be incorporated at levels of up to 5% by weight. Film formers are polymers which are often incorporated into skin care formulations, and function to form a continuous film on the surface of the skin, providing a protective layer, and also assisting in the retention of moisture. There are a large number of film formers available in the trade, depending on the desired features of flexibility, water-repellency, staying power, and permeability. Suitable film formers used in formulations of the invention included GLYCOFILM 1.5P® (main ingredient: biosaccharide gum-4) and FUCOGEL® powder (main ingredient: biosaccharide gum-1) both of which are available from SOLABIA®. These film formers are also known to provide some soothing effect to the skin, and provide a smooth after-feel to the formulation upon application.

If a film former is used in the formulation, it may be incorporated into Phase C after addition of the lipophilic peptide, during the 3-5 hours of agitation at 3,000 RPM with a side sweep at 10 RPM, as described previously.

A sample formulation for Phase C is shown in Table 1.

TABLE 1

| Phase C Ingredient | % (w/w) in final formulation |
|---|---|
| RHEANCE ONE (glycolipids) | 5.00% |
| LANABLUE (algae extract) | 5.00% |
| VETIVYNE (vetiver extract) | 3.00% |
| Pal-GHK-Cu (peptide) | 1.00% |

Phase A of the composition comprises a thickening component and emulsifier which facilitates the formation of a gel. Many options are available, but a suitable and beneficial choice was found to be SUGRAGEL XL®, which is marketed by ALFA CHEMICALS®. SUCRAGEL XL is a mixture of ingredients having skin care benefits, in addition to acting as a thickener and emulsifier during formulation. SUCRAGEL XL is made up of glycerin (an emollient and humectant); caprylic and capric triglycerides (moisturizers and general skin conditioners); water, and sucrose laurate and sucrose stearate (both effective skin emollients and conditioners).

In phase A, SUCRAGEL XL may be used in an amount in the range of 15-25% in accordance with the invention. A preferred amount is 20%.

To prepare phase A, the SUCRAGEL XL is added into a mixing vessel and mixed for 10 minutes at 800-1000 RPM, using a side sweep speed of 10 RPM, at room temperature. It can be maintained under these conditions while preparing phase B. A sample formulation for phase A is shown in Table 2.

TABLE 2

| Phase A Ingredient | % (w/w) in final formulation |
|---|---|
| SUCRAGEL XL (glycerin, | 20.00% |
| caprylic/capric triglyceride, water, | |
| sucrose laurate, sucrose stearate) | |

Phase B of the invented composition comprises other beneficial skin active ingredients. Phase B comprises oil-based ingredients and includes MCT oil (Medium Chain Triglycerides, also known as caprylic/capric triglycerides or CCTG). Squalane is also used to contribute to the base, and an additional active ingredient, SYM3D®, is used in phase B.

Phase B may be prepared by adding MCT oil into a mixing vessel, and incorporating further ingredients such as one or more oil-soluble active ingredients. The ingredients are mixed at room temperature at a speed of 3000 RPM, with side sweep scraping at 15 RPM. Samples should be taken via the valve at the bottom of the kettle and tested by centrifugation, and the solution also visibly inspected to ensure complete incorporation of the second ingredient. Centrifugation of a sample of the mixture is an optional step to ensure complete dissolution of the second ingredient.

The MCT oil used is technically an ester. It is a commonly used ingredient for personal care formulations, as it is an effective skin conditioner. This product is available from many suppliers. The inventors used C-SYN-MCT® marketed by CUSTOM SYNTHESIS, LLC®. An amount of MCT oil suitable to result in a level of 10% in the final formulation was used. Amounts ranging from 10-20% MCT oil would be workable in the formulation.

One active ingredient that may be mixed into the MCT oil is SYM3D®, marketed by SYMRISE AG®. SYM3D is the trade name for dihydrodehydrodiisoeugenol, having an INCI name of hydroxymethoxyphenyl propylmethylmethoxybenzofuran. It presents as a hydrophobic white powder. SYM3D has been promoted by SYMRISE as stimulating lipid uptake and accumulation in adipocytes as well as increasing adipogenesis and adipocyte size, which ultimately results in more youthful looking skin. SYM3D also has the additional benefit of antioxidative activity.

Amounts of SYM3D that would be effective in the invented formulation range from 0.1 to 1% by weight of the overall formulation. The specific amount used by the inventors in a sample formulation was 0.5%. SYM3D is selectively soluble in certain oils. It can be solubilized in MCT oil in the conditions as described above, but for example it is not soluble in squalane. It is therefore important to include an oil component such as MCT oil which is compatible with SYM3D if it is being used.

A further ingredient that may also be added to the MCT oil at or around the same time as SYM3D is SYMRELIEF S®, which is a soothing ingredient made up of a mix of bisabolol and hydroxymethoxyphenyl decanone, and which is also marketed by SYMRISE AG. It is a blend of anti-irritants which also provides antioxidant activity, and is therefore a further beneficial ingredient for the skin. SYMRELIEF S may be incorporated, if used, in an amount of approximately 0.1% by weight. Many other soothing additives are available in the trade and may be used.

Following addition and the complete incorporation of any oil-soluble active ingredients to the MCT oil, a further oil phase ingredient, squalane purchased from NEOSSANCE®, was added slowly using gentle agitation at room temperature, using only the side sweep scrapers at a speed of 25 RPM for 10 minutes. Squalane is an effective emollient and humectant which is commonly used in skin care formulations. The inventors used it in an amount resulting in a range of 55-60% of the formulation overall, but lower amounts would also be effective.

A sample formulation for phase B is shown in Table 3.

TABLE 3

| Phase B Ingredient | % (w/w) in final formulation |
|---|---|
| C-SYN MCT (caprylic capric | 10.00% |
| triglycerides) | |
| SYM 3D (hydroxymethoxyphenyl | 0.50% |
| propylmethylmethoxybenzofuran) | |
| NEOSSANCE SQUALANE | 55.50% |

Following preparation of phases A, B, and C as outlined above, the phases can be combined as follows. First, Phase B should be slowly added into Phase A while it is being stirred at 800-1000 RPM. At this point, the side sweep scrapers are activated at a speed of 15 RPM. Approximately 10% of Phase B should be added at first, with visual monitoring of the mixture to avoid pooling. Additional amounts of Phase B may then be progressively added while continuing to avoid pooling. Both the side sweep and homogenizer speeds can be gradually increased to 50 RPM and 3000 RPM respectively while monitoring the incorporation of Phase B into Phase A. It is necessary to increase the homogenizer speeds gradually because the product becomes more viscous and harder to stir as the levels rise in the kettle. Once Phase B addition is complete, mixing should continue at 50 RPM and 3000 RPM respectively for the side sweep and homogenizers, for 30 minutes at room temperature.

The next step is the addition of Phase C to the mixture of Phase A/B. Phase C should also be incorporated slowly at a rate of approximately 10% of its volume at a time, to the Phase A/B mixture while stirring at 50 RPM side sweep and homogenizer speeds at 3000 RPM. Once all of Phase C has been added, mixing at these speeds continues for 30 minutes at room temperature. It is important at this stage to also activate the cooling jacket on the kettle in order to maintain the mixture at room temperature levels. As the mixture continues to get agitated and the homogenizer speeds increase, the temperature in the kettle will tend to rise. Careful monitoring of temperature levels and cooling therefore becomes necessary so that the delicate peptides are not compromised.

An optional phase D may also be included in the process. Phase D may consist of quantity of preservative. A suitable preservative for use in the formulations is ISCAGUARD CPP®, a well-known cosmetic preservative available from ISCA UK®. ISCAGUARD CPP is made up of chlorphenesin and phenoxyethanol, and may be used in an amount in the range of 0.50%. Phase D may be added at the latter stages of the incorporation of Phase C into Phase A/B as described above.

A sample formulation for Phase D is shown in Table 4.

TABLE 4

| Phase D Ingredient | % (w/w) in final formulation |
|---|---|
| ISCAGUARD CPP (phenoxyethanol, | 0.50% |
| chlorphenesin) | |

FIG. 2 shows a schematic of the core phases A-C of the manufacturing process outlined above.

Table 5 shows sample detailed formulations.

TABLE 5

| | Percentage | Formulation | Formulation | Formulation | Formulation |
|---|---|---|---|---|---|
| | Ranges* | 21-001 | 21-209 | 21-224 | 21-254 |
| Ingredient | (w/w) | (w/w) | (w/w) | (w/w) | (w/w) |
| SUCRAGEL XL (glycerin, caprylic/ | 15-25% | 20.00% | 20.57% | 21.45% | 21.57% |
| capric triglyceride, water, sucrose | | | | | |
| laurate, sucrose stearate) | | | | | |
| C-SYN MCT (caprylic capric | 10.0-20.0% | 10.00% | 10.00% | 10.00% | 10.00% |
| triglycerides) | | | | | |
| SYM 3D (hydroxymethoxyphenyl | 0-1.0% | 0.50% | 0.50% | 0.50% | 0.50% |
| propylmethylmethoxybenzofuran) | | | | | |
| NEOSSANCE SQUALANE | 20.0-65.5% | 55.50% | 56.08% | 56.96% | 57.08% |
| RHEANCE ONE (glycolipids) | 1-6% | 5.00% | 1.25% | 1.25% | 1.25% |
| LANABLUE (algae extract) | 0-5% | 5.00% | 5.00% | 5.00% | 5.00% |
| VETIVYNE (vetiver extract) | 0-3% | 3.00% | 3.00% | 3.00% | 3.00% |
| Pal-GHK-Cu (peptide) | 0.2-1.1% | 1.00% | 1.00% | 1.00% | 1.00% |
| SYMRELIEF S | 0-0.2% | — | 0.10% | 0.10% | 0.10% |
| GLYCOFILM 1.5P | 0-5% | — | 2.00% | — | — |
| FUCOGEL POWDER | 0-1% | — | — | 0.24% | — |
| ISCAGUARD CPP | 0-1% | — | 0.50% | 0.50% | 0.50% |

*suitable amounts to be used to add up to 100%

The formulation may further include additional ingredients, subject to solubility in the formulation components. Such additional ingredients may include cosmetic ingredients that enhance or supplement a lipophilic peptide formulation. The term “cosmetic ingredient” is used herein to refer to a product for application to the body (e.g. skin, hair, or nails) to improve or enhance some feature of the body, such as by providing skincare benefits. Thus, additional cosmetic ingredients may be included such as conditioners, emollients, emulsifiers, humectants, botanical extracts, natural oils, silicones, sunscreens, surfactants and thickeners. Other examples of cosmetic ingredients include, but are not limited to, other peptides; amino acids (e.g. serine, lysine, proline, glycine, glutamine); carbohydrates (e.g. ascorbyl glucoside, glucosyl hesperidin, and saccharide isomerate); proteins (e.g. collagen, elastin, and hydrolyzed lupine protein); lipids (e.g. ceramides, glycosphingolipids, and phospholipids); vitamins (e.g. niacinamide, biotin, ascorbic acid, retinol, retinoic acid, and panthenol); sterols (e.g. phytosterols, punica granatum sterols, and 7-dehydrocholesterol); flavanoids (e.g. apigenin, heseperidin, and genistein); esters (e.g. methylsilanol mannuronate, butyl avocadoate, and jojoba esters); inorganics (e.g. kaolin, zinc and magnesium); botanical products (e.g. Tasmannia Lanceolata fruit/leaf extract, Curculigo Orchioides root extract, and rose flora); and biotechnological products (e.g. Pseudoalteromonas exopolysaccharides, Alteromonas ferment filtrate, sodium hyaluronate).

The formulation of the invention may also contain vitamins and derivatives thereof such as panthenol, ascorbic acid, niacinamide, tocopherol; anti-inflammatory ingredients such as allantoin, phytantriol, sphingosine and bisabolol; other moisturizing ingredients such as trimethylglycine and polyglutamic acid; additional antioxidants such as flavonoids, xanthones, isoflavones, alpha-lipoic acid; and other anti-aging actives such as beta-glucans, alpha hydroxy acids, beta hydroxy acids.

The formulation may also include fragrance such as essential oils, plant extracts, or synthetic fragrance. Examples include melon, vanilla, cucumber, aloe vera, almond, mango, coconut, cocoa butter, shea butter, linalool, citronellol, cinnamal, limonene, geraniol, eugenol, lavender oil, rose flower extract, bergamot oil, ylang-ylang oil, lemon, lime, orange, tangerine, peppermint, spearmint, and eucalyptus.

As used herein, the term “about” refers to an amount within 10% or less of the indicated amount, and preferably an amount within 5% or less, wherein the amount is either greater or less than the indicated amount.

The present invention therefore corresponds to a skin care formulation containing about 0.2-1.1% of a lipophilic peptide in solution. Preferred lipophilic peptides for use in the invention are Pal-GHK-Cu and Pal-GHK. An especially preferred amount of the lipophilic peptide is 1%.

The present invention also corresponds to a premix of a lipophilic peptide in a glycolipid solution which is free of alcohol or other harsh solvents, and which facilitates the dissolution of the peptide at the concentrations described above. The premix utilizes quantities of glycolipids to peptide which will result in a concentration by weight of 1-6% glycolipids and 0.2-1.1% peptide in the final formulation. In the premix, these ranges correspond to a mixture of 3.2 to 52.4% peptide w/w dissolved in glycolipids. Particularly preferred are the amounts shown in the sample formulations. When 1% peptide and 1.25% glycolipids are used in the premix which forms part of phase A, this corresponds to 44% peptide w/w dissolved in glycolipids. When 1% peptide and 5% glycolipids are used in the premix which forms part of phase A, this corresponds to 17% peptide w/w dissolved in glycolipids.

The present invention also corresponds to a skin care formulation containing, as percentages by weight of the final formulation:

(a) a lipophilic peptide, preferably Pal-GHK-Cu or Pal-GHK, most preferably Pal-GHK-Cu, in the amount of 0.2-1.1%;
(b) glycolipids in the amount of 1-6%;
(c) a thickener in the amount of 15-25%;
(d) caprylic capric triglycerides in the amount of 10.0-20.0%;
(e) the balance being squalane, the proviso being that (a) to (e) must add up to 100%.

The present invention also corresponds to a skin care formulation containing, as percentages by weight of the final formulation:

(a) a lipophilic peptide, preferably Pal-GHK-Cu, in the amount of 1.0%;
(b) glycolipids in the amount of 1.25-5.00%;
(c) a thickener in the amount of 20.00-21.57%;
(d) caprylic capric triglycerides in the amount of 10.00%;
(e) an antioxidant, preferably hydroxymethoxyphenyl propylmethylmethoxybenzofuran, in the amount of 0.50%;
(f) an algal extract additive in the amount of 5.00%;
(g) a vetiver extract additive in the amount of 3.00%;
(h) the balance being squalane, the proviso being that (a) to (h) must add up to 100%.

The present invention also corresponds to a skin care formulation containing, as percentages by weight of the final formulation:

(a) a lipophilic peptide, preferably Pal-GHK-Cu, in the amount of 1.0%;
(b) glycolipids in the amount of 1.25-5.00%;
(c) a thickener in the amount of 20.00-21.57%, with particularly preferred amounts being 20.00%, 20.57%, 21.45%, and 21.57%;
(d) caprylic capric triglycerides in the amount of 10.00%;
(e) an antioxidant, preferably hydroxymethoxyphenyl propylmethylmethoxybenzofuran, in the amount of 0.50%;
(f) an algal extract additive in the amount of 5.00%;
(g) a vetiver extract additive in the amount of 3.00%;
(h) a soothing agent in the amount of 0.1-0.2%; preferably 0.10%;
(i) a preservative in the amount of 0.1-1.0%; preferably 0.50%;
(h) the balance being squalane, the proviso being that (a) to (h) must add up to 100%.

The present invention also corresponds to a skin care formulation containing, as percentages by weight of the final formulation:

(a) a lipophilic peptide, preferably Pal-GHK-Cu, in the amount of 1.0%;
(b) glycolipids in the amount of 1.25-5.00%, preferably 1.25%;
(c) a thickener in the amount of 20.00-21.57%, with particularly preferred amounts being 20.57%, 21.45%;
(d) caprylic capric triglycerides in the amount of 10.00%;
(e) an antioxidant, preferably hydroxymethoxyphenyl propylmethylmethoxybenzofuran, in the amount of 0.50%;
(f) an algal extract additive in the amount of 5.00%;
(g) a vetiver extract additive in the amount of 3.00%;
(h) a soothing agent in the amount of 0.1-0.2%, preferably 0.10%;
(i) a film former in the amount of 0.1-5%, preferably 0.24-2.00%;
(j) a preservative in the amount of 0.1-1.0%; preferably 0.50%;
(k) the balance being squalane, the proviso being that (a) to (k) must add up to 100%.

Preparation of the formulation in accordance with the methods described herein results in a transparent blue, oil-based gel when Pal-GHK-Cu is used, and a translucent oil-based gel when Pal-GHK is used. As it contains peptides which are delicate ingredients sensitive to degradation by light, the formulation should be packaged in opaque packaging, such as an aluminum squeeze tube.

The formulation is used on the skin after cleansing by rubbing a small amount directly onto the skin. Another unique aspect of this formulation is that the gel turns into a creamy emulsion upon addition of body heat from the hand and rubbing onto the skin, without any oily feel.

### Water Activity Testing

As set out in Table 5 and described above, the invented formulation consists primarily of ingredients that have benefits to the skin. The formulation also lacks the usual requirement for extraneous preservatives such as triclosan, benzyl benzoate, methylisothiazolinone, or parabens. Typically, such preservatives are required to discourage bacterial growth and to prevent degradation of the delicate peptides incorporated in the formulation. However, as detailed below, the formulation has been configured as a low water-activity gel which does not require preservatives. This is highly beneficial as it means that the formulation may consist almost fully of skin-active and beneficial ingredients, without levels of preservatives that can interfere with skin biota or otherwise have drying or other negative effects.

Water Activity, expressed as K_{w} or A_{w}, is a standard measure used with personal care formulations to estimate shelf life and to determine whether preservatives will be required. It is not the same as water content. Rather, the test measures the bioavailability of water for biological reactions by micro-organisms such as gram positive and gram negative bacteria, yeast, or fungi. For reference, bone powder has a water activity of 0. Pure water has a water activity of 1.0.

In the cosmetics field, a formulation having a water activity of less than 0.7 indicates that there is no need for a preservative.

The water activity of the invented formulations were tested using a standard water activity testing instrument, the AQUALAB® TDL. The water activities for the formulations were less than 0.65, indicating that the formulations do not require preservatives. This is a further advantage of offering this peptide formulation as a gel, which not only lowers the water activity and obviates the need for a preservative, but also provides for conditions which stabilize the delicate peptides incorporated therein. In the industry, most peptides are offered in serum form which tend to have high levels of water activity and thus may require either cold storage conditions or the addition of significant amounts of preservatives.

It is noted that some ingredients suitable for optional incorporation into the formulation of the invention may contain small amounts of preservatives to protect the active ingredient. For instance, LANABLUE, whose active ingredient is algal extract, also contains small amounts of phenoxyethanol and potassium sorbate which are preservatives. However, ingredients like LANABLUE are optional so it is feasible to make a version without this ingredient.

The formulations may also be prepared to include added preservatives, such as the ISCAGUARD CPP present in some of the sample formulations. Despite the low water activity, it may be beneficial to include an amount of preservative in any event as additional assurance that the formulation will remain free of contamination. However, an added preservative is not required.

The formulations of the invention also do not contain any extraneous alcohols or solvents, and certainly not at the levels that would normally be required to dissolve lipophilic ingredients like the peptides used herein. However, certain optional ingredients may themselves contain small amounts of alcohol. For instance, VETIVYNE contains a small amount of propanediol. Again, VETIVYNE is an optional ingredient so it is entirely feasible to omit or substitute this ingredient if it is desired to make a completely alcohol-free embodiment.

### Other Testing

The formulation of the invention is also considered non-sensitizing or irritating to skin. Standard cosmetic patch testing in accordance with an occlusive patch testing protocol was performed on fifty volunteers, with test product being applied and kept in contact with the skin for 48 hours under an occluded patch. No adverse events were observed. The Mean Irritation Index level, a measure of whether a product is irritating or sensitizing to the skin, was found to be “NOT IRRITANT”.

Standard testing for stability of formulations of the invention was also performed. Aliquots of the formulations were tested under conditions of 45° C., room temperature, 4° C., −25° C., and under conditions of UV exposure. The testing results indicated a 12 month shelf life under normal storage conditions, which is an acceptable duration for such cosmetic formulations.

Standard testing for comedogenicity on forty volunteers has also confirmed that the formulations are non-comedogenic.

The invention has therefore been confirmed to provide a high-concentration lipophilic peptide solution in a formulation that may be substantially free of preservatives and alcohols or other harsh solvents. The methods described herein, particularly the method for solubilizing the lipophilic peptide to result in high concentrations, are novel and result in a formulation with an approximately ten fold increase in peptide concentration as compared to existing solutions, with benefits for the skin as described herein. The formulation is further useful as a cosmetic for skin, being non-irritating and non-comedogenic, and having sufficient shelf life for a commercial product.

Various embodiments of the invention have been described. These and other embodiments are within the scope of the following claims.

1. A skin care formulation containing 0.2-1.1% of a lipophilic peptide in solution, by weight, based on total weight of the formulation.

2. The skin care formulation of claim 1 in which the lipophilic peptide is Pal-GHK-Cu or Pal-GHK.

3. The skin care formulation of claim 1 in which the lipophilic peptide is provided in the amount of 1%.

4. The skin care formulation of claim 1 further comprising by weight, based on the total weight of the formulation:
(a) glycolipids in the amount of 1-6%;
(b) a thickener in the amount of 15-25%;
(c) caprylic capric triglycerides in the amount of 10.0-20.0%; and
(d) squalane q. s.

5. The skin care formulation of claim 4 further comprising by weight, based on the total weight of the formulation:
(a) an antioxidant, in the amount of 0.50%;
(b) an algal extract additive in the amount of 5.00%; and
(c) a vetiver extract additive in the amount of 3.00%.

6. The skin care formulation of claim 5 in which the antioxidant is hydroxymethoxyphenyl propylmethylmethoxybenzofuran.

7. The skin care formulation of claim 5, further comprising by weight, based on the total weight of the formulation:
(a) a soothing agent in the amount of 0.1-0.2%; and
(b) a preservative in the amount of 0.1-1.0%;

8. The skin care formulation of claim 7, in which
(a) the soothing agent is provided in the amount of 0.10%; and
(b) the preservative is provided in the amount of 0.50%.

9. The skin care formulation of claim 1, which is substantially free of alcohols.

10. The skin care formulation of claim 1, which has a water activity level (A_{W}) of 0.65 or lower.

11. A mixture of 3.2-52.4% (w/w) of a lipophilic peptide dissolved in glycolipids.

12. The mixture of claim 11, in which the amount of lipophilic peptide is 17-44%.

13. Use of the mixture of claim 11 to formulate a skin care composition.

14. A method for manufacturing a skin care composition, comprising:
(a) Preparation of the mixture of claim 11 to form a first component;
(b) Agitation of a thickener to form a second component;
(c) Blending caprylic capric triglycerides with squalane to form a third component;
(d) Combining said third component with said second component under agitation to form a first combination; and
(e) Combining said first component with said first combination under agitation to form a second combination.

15. The method of claim 14, further comprising
(f) Combining said second combination with a preservative.

16. Use of the method of claim 14 to formulate the skin care composition of claim 4.
